(19) United States
(12) Patent Application Publication
(10) Pub. No.: US 2015/0094545 A1
(43) Pub. Date: Apr. 2, 2015

(54) AUTOMATED AT-REST STATUS SENSING

(71) Applicant: Covidien LP, Mansfield, MA (US)

(72) Inventors: BRIAN KEITH RUSSELL, Annapolis, MD (US); JONATHAN JAMES WOODWARD, Annapolis, MD (US); BENJAMIN DAVID MORRIS, Annapolis, MD (US)

(21) Appl. No.: 14/497,824

(22) Filed: Sep. 26, 2014

Related U.S. Application Data
(60) Provisional application No. 61/885,182, filed on Oct. 1, 2013.

(51) Int. Cl. A61B 5/00; A61B 5/0488; A61B 5/145; A61B 5/0402; A61B 5/11; A61B 5/0205; A61B 5/0476

(52) U.S. Cl. CPC A61B 5/7221; A61B 5/02055; A61B 5/0022; A61B 5/0488; A61B 5/0476; A61B 5/0402; A61B 5/1118; A61B 5/1116; A61B 5/14532; A61B 5/14542; A61B 5/4266; A61B 5/6802; A61B 5/7282; A61B 5/021
USPC 600/301

(57) ABSTRACT

Methods, apparatuses and systems are described for associating remote physiological monitoring with an at-rest condition of a patient. The methods may include receiving activity data and physiological data of a patient. The methods may also include determining that an at-rest condition is satisfied by at least one of the received activity data and physiological data. Once it is determined that the at-rest condition is satisfied, the methods may also include associating an at-rest indicator with one or more physiological measurements of the patient.

### CROSS-REFERENCE TO RELATED APPLICATIONS

This application claims priority to U.S. Provisional Patent Application No. 61/885,182, filed on Oct. 1, 2013, the entirety of which is incorporated by reference herein.

### BACKGROUND

The present disclosure relates generally to physiological monitoring systems, and more particularly to associating physiological measurements with an at-rest status of a patient.

At-rest physiological measurements are used by clinicians for a variety of clinical purposes, including determining fitness level, detecting cardiac disease, and monitoring stress level. Collecting at-rest physiological measurements, however, may be difficult and unreliable. Traditionally, at-rest physiological measurements are taken in a clinical setting, where a clinician may ask the patient to sit or lie down in order to facilitate an at-rest status in the patient. Given the limited time during which a clinician is available to see each patient, however, the patient may not have ample time to achieve an at-rest status such that the physiological measurements are truly taken at-rest. Additionally, such methods limit the clinician to brief snapshots of physiological measurements based on the patient's limited visits to the office, with no data gathered relating to the patient's physiological measurements during the time the patient is away from the doctor's office.

One way to collect physiological measurements is to provide a patient with a remote monitoring device. Traditional remote monitoring devices, however, may monitor and/or transmit physiological measurements constantly, with no means provided to differentiate between physiological measurements gathered when the patient is active and physiological measurements gathered when the patient is at-rest. Thus, for example, physiological measurements transmitted to a clinician from these remote devices might indicate an increased heart rate but could fail to indicate whether the increased heart rate was due to exercise or some other activity, or whether the increased heart rate was indicative of some other underlying concern. For example, the increased heart rate could be due to infection, or could be merely due to a patient's normal workout session. The clinician, however, may have no means by which to differentiate between the two possible causes or any other cause. The physiological measurements provided by the remote monitoring device may therefore be largely medically irrelevant, and may lead to misdiagnosis.

In order to remotely monitor physiological measurements of a patient, it is therefore necessary to provide a means by which it may be determined that the patient is at-rest, and a means to correlate the physiological measurements transmitted to the caregiver with these periods of patient at-rest status.

### SUMMARY

Because many physiological measurements are only clinically relevant when taken while the patient is at-rest, it may be beneficial to a clinician to receive remotely monitored physiological measurements “flagged” as having been taken during an at-rest period. In this way, the clinician may be able to identify variations in physiological measurements, for example heart rate, which cannot be attributed to normal patient activity or non-life threatening anxiety, and may therefore be attributable instead to some underlying health issue. One method of accomplishing this includes receiving activity data of a patient from one or more sensors, receiving physiological data of the patient from one or more sensors, and determining that an at-rest condition of the patient is satisfied by at least one of the received activity data and physiological data. An at-rest indicator may then be associated with one or more physiological measurements of the patient based at least in part on this determination.

By collecting both activity data and physiological data in order to determine that an at-rest condition is satisfied, a patient may be determined to be at-rest based on either the activity data or the physiological data, or alternatively, a patient may be determined to be at-rest based on both the activity data and the physiological data such that situations in which a patient is at-rest mechanically, but not at-rest physiologically, or vice versa, may be disregarded. For example, a patient who has recently run three miles and is now seated may be mechanically at-rest, but his heart rate may continue to be elevated as a result of the physical exertion for an additional five minutes after he sits down, such that any physiological measurements collected during this period of “recovery” are not related to a true at-rest status. Thus, by requiring that an at-rest condition be satisfied by both physiological data and activity data, a clinician may decipher those physiological measurements collected when the patient is truly at-rest.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

Further scope of the applicability of the described methods and apparatuses will become apparent from the following detailed description, claims, and drawings. The detailed description and specific examples are given by way of illustration only, since various changes and modifications within the spirit and scope of the description will become apparent to those skilled in the art.

### BRIEF DESCRIPTION OF THE DRAWINGS

A further understanding of the nature and advantages of the present invention may be realized by reference to the following drawings. In the appended figures, similar components or features may have the same reference label. Further, various components of the same type may be distinguished by following the reference label with a dash and a second label that distinguishes among the similar components. If only the first reference label is used in the specification, the description is applicable to any one of the similar components having the same first reference label irrespective of the second reference label.

FIG. 1 is a block diagram of an example of a physiological monitoring system in accordance with various embodiments;

FIG. 2 is a graphical representation of the determination of at-rest status in relation to physiological data and activity data received in accordance with various embodiments;

FIG. 3 is a block diagram of an example of an apparatus in accordance with various embodiments;

FIG. 4 is a block diagram of an example of an apparatus in accordance with various embodiments;

FIG. 5 is a block diagram of an example of a sensing apparatus for receiving physiological data and activity data in accordance with various embodiments;

FIG. 6 is a block diagram of an example of a server for determining at-rest status in accordance with various embodiments; and

FIGS. 7 and 8 are flowcharts of various methods for determining at-rest status in accordance with various embodiments.

### DETAILED DESCRIPTION

In order to efficiently understand the physiological condition of a patient, clinicians monitor a plurality of physiological measurements of the patient. These measurements may include, for example, the patient's heart rate, heart rate variability, respiration rate, respiration rate variability, blood pressure, oxygen saturation levels, glucose levels, weight, perspiration, core temperature, electromyography data, electroencephalogram data, etc. These physiological measurements may only serve as accurate indicators of a patient's health, however, when monitored when the patient is at-rest. For example, increased perspiration may be a natural byproduct of vigorous exercise in a healthy patient, but may be indicative of thyroid issues, infection, or diabetes in an unhealthy patient when observed when the patient is at-rest. Presenting physiological measurements to a clinician flagged as having been taken when the patient was at-rest, therefore, may provide the clinician with valuable background information as to the medical relevance of the measurements received.

An at-rest status may be achieved by a patient being physiologically at-rest and/or mechanically at-rest. For example, a patient may be mechanically at-rest in that the patient is seated, but may not be physiologically at-rest because he has just climbed a flight of stairs to reach the chair in which he is seated, and his heart rate and respiration rate may therefore still be elevated. Thus, the combination of mechanical at-rest status and physiological at-rest status may best indicate to a clinician that physiological measurements taken during a particular period of time are taken when the patient is truly at-rest.

The present disclosure includes a method and system for determining and presenting to the clinician physiological measurements associated with flags indicating which physiological measurements were taken when the patient was mechanically and/or physiologically at-rest. The recorded physiological measurements may be collected through a physiological monitoring system. One example of a physiological monitoring system is a remote physiological monitoring system. Examples below describe such a system, though it should be understood that any type of physiological monitoring system may provide physiological data and activity data from which an at-rest status may be determined for display to a clinician.

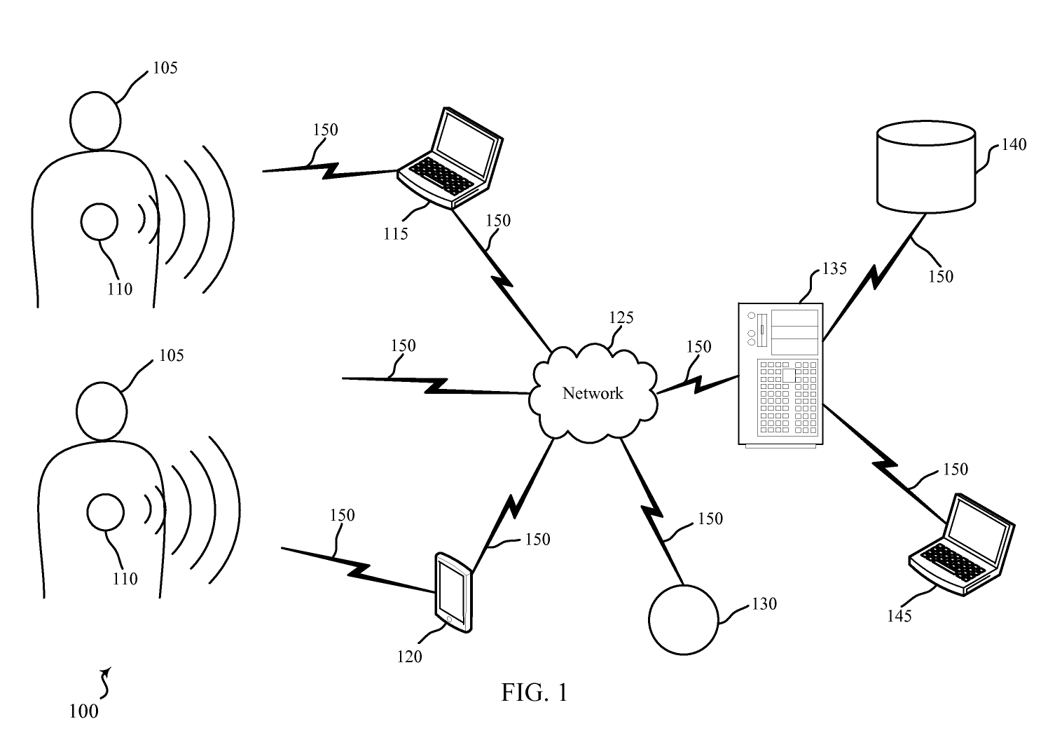

Referring first to FIG. 1, a diagram illustrates an example of a remote physiological measurement monitoring system 100. The system 100 includes patients 105, each wearing a sensor unit 110. The sensor units 110 transmit signals via wireless communication links 150. The transmitted signals may be transmitted to local computing devices 115, 120. Local computing device 115 may be a local caregiver's station, for example. Local computing device 120 may be a mobile device, for example. The local computing devices 115, 120 may be in communication with a server 135 via network 125. The sensor units 110 may also communicate directly with the server 135 via the network 125. Additional, third-party sensors 130 may also communicate directly with the server 135 via the network 125. The server 135 may be in further communication with a remote computing device 145, thus allowing a caregiver to remotely monitor the patients 105. The server 135 may also be in communication with various medical databases 140 where the collected data may be stored.

The sensor units 110 are described in greater detail below. The sensor units 110 may be a body-worn device, coupled to the patient's chest or to any other suitable portion of the patient's body, such as the patient's arm, wrist, or thigh. The sensor units 110 may be coupled to the patient using an adhesive, a strap, or any other suitable means. In an alternative embodiment, the sensor units 110 may be coupled to or integral with a garment worn by a patient, such as a belt, wristband, headband, armband, or piece of clothing.

In some embodiments, the sensor units 110 are sensors configured to conduct periodic or ongoing automatic measurements of one or more physiological measurements, physiological data and/or activity data. A patient may wear or otherwise be attached to one or more sensor units 110 so that the sensor units 110 may measure, record, and/or report physiological measurements, physiological data, and/or activity data associated with the patient.

Each sensor unit 110 may be capable of sensing multiple physiological measurements, as well as sensing physiological data and activity data. Thus, the sensor units 110 may each include multiple sensors such as heart rate and ECG sensors, respiratory rate sensors, and accelerometers. For example, a first sensor in a sensor unit 110 may be an oxygen saturation monitor or a glucose level monitor operable to detect a patient's blood oxygen or sugar levels. A second sensor within a sensor unit 110 may be operable to detect a second physiological parameter. For example, the second sensor may be a heart rate monitor, an electrocardiogram (ECG) sensing module, a breathing rate sensing module, and/or any other suitable module for monitoring any suitable physiological measurement. A third sensor within a sensor unit 110 may be operable to detect the position, velocity, and/or acceleration of the sensor unit 110. Multiple sensor units 110 may be used on a single patient 105. The data collected by the sensor units 110 may be wirelessly conveyed to either the local computing devices 115, 120 or to the remote computing device 145 (via the network 125 and server 135). Data transmission may occur via, for example, frequencies appropriate for a personal area network (such as Bluetooth or IR communications) or local or wide area network frequencies such as radio frequencies specified by the IEEE 802.15.4 standard.

The sensor units 110 may include any of the sensors, detectors, and/or modules operable to detect physiological parameters illustrated and described in U.S. Patent Publication No. 2011/0257542, filed Apr. 15, 2011; U.S. Patent Publication No. 2012/0143019, filed Jun. 6, 2011; U.S. Patent Publication No. 2009/0227856, filed Dec. 19, 2008; U.S. Patent Publication No. 2009/0281394, filed Sep. 21, 2007; U.S. Patent Publication No. 2013/0144130, filed Jan. 30, 2012; U.S. Patent Application No. 61/823,596, filed Mar. 15, 2013; U.S. Patent Application No. 61/864,161, filed Aug. 9, 2013; U.S. Pat. No. 8,400,302, issued Mar. 19, 2013; and/or U.S. Pat. No. 8,079,247, issued Dec. 20, 2011, each of which is commonly owned and which is incorporated herein by reference in its entirety.

In one embodiment, one or more sensor units 110 comprises an accelerometer to measure patient activity data. The accelerometer may be a three-axis microelectromechanical system (MEMS) accelerometer, a piezoelectric accelerometer, a mechanical accelerometer, and/or any other suitable device to detect acceleration and/or static acceleration fields (e.g., the gravitational field). In addition or alternatively, the accelerometer may include a gyroscope operable to detect angular position, angular velocity, and/or angular acceleration of the sensor unit 110.

In some embodiments, one or more third-party sensors 130 may comprise sensors to detect environmental data, such as surface pressure to detect whether a patient 105 is sitting down or lying still. Additionally, one or more third-party sensors 130 may comprise video cameras for detecting whether the patient 105 is sitting down or lying still. In this way, the third-party sensors 130 may provide data relating to the activity data of the patient 105. In some embodiments, third-party sensors 130 may detect environmental data such as temperature, humidity or vibration data in order to determine patient activity data or physiological data. For example, if third-party sensor 130 detects increased humidity, this data may be indicative of increased patient respiration and body temperature. In another example, if third-party sensor 130 detects vibration data, this data may be indicative of patient movement or activity.

The local computing devices 115, 120 may enable the patient 105 and/or a local caregiver to monitor the collected physiological measurements. For example, the local computing devices 115, 120 may be operable to present data collected from sensor units 110, 130 in a human-readable format. For example, the received data may be outputted as a display on a computer or a mobile device. The local computing devices 115, 120 may include a processor that may be operable to present data received from the sensor units 110, 130 in a visual format. The local computing devices 115, 120 may also output data in an audible format using, for example, a speaker. In alternative embodiments, the received data may be output as a display or in an audible format from the one or more sensor units 110 themselves.

The local computing devices 115, 120 may be custom computing entities configured to interact with the sensor units 110. In some embodiments, the local computing devices 115, 120 and the sensor units 110 may be portions of a single sensing unit operable to sense and display physiological measurements. In another embodiment, the local computing devices 115, 120 may be general purpose computing entities such as a personal computing device, for example a desktop computer, a laptop computer, a netbook, a tablet personal computer (PC), an iPod®, an iPad®, a smart phone (e.g., an iPhone®, an Android® phone, a Blackberry®, a Windows® phone, etc.), a mobile phone, a personal digital assistant (PDA), and/or any other suitable device operable to send and receive signals, store and retrieve data, and/or execute modules.

The local computing devices 115, 120 may include memory, a processor, an output, a data input and a communication module. The processor may be a general purpose processor, a Field Programmable Gate Array (FPGA), an Application Specific Integrated Circuit (ASIC), a Digital Signal Processor (DSP), and/or the like. The processor may be configured to retrieve data from and/or write data to the memory. The memory may be, for example, a random access memory (RAM), a memory buffer, a hard drive, a database, an erasable programmable read only memory (EPROM), an electrically erasable programmable read only memory (EEPROM), a read only memory (ROM), a flash memory, a hard disk, a floppy disk, cloud storage, and/or so forth. In some embodiments, the local computing devices 115, 120 may include one or more hardware-based modules (e.g., DSP, FPGA, ASIC) and/or software-based modules (e.g., a module of computer code stored at the memory and executed at the processor, a set of processor-readable instructions that may be stored at the memory and executed at the processor) associated with executing an application, such as, for example, receiving and displaying data from sensor units 110.

The data input module of the local computing devices 115, 120 may be used to manually input physiological measurements instead of or in addition to receiving data from the sensor units 110. For example, a user of the local computing device 115, 120 may make an observation as to one or more physiological or activity conditions, or physiological measurements, of a patient and record the observation using the data input module. A user may be, for example, a nurse, a doctor, and/or any other medical healthcare professional authorized to record patient observations, the patient, and/or any other suitable person. For instance, the user may measure the patient's body temperature (e.g., using a stand-alone thermometer) and enter the measurement into the data input module. In some embodiments, the data input module may be operable to allow the user to select “body temperature” and input the observed temperature into the data input module, e.g., using a keyboard. In other embodiments, the data input module may be operable to allow the user to select whether the patient is stationary or is mechanically in motion. Automatically collected physiological data and activity data may be used to flag the manually input physiological measurements as being associated with an at-rest status.

The processor of the local computing devices 115, 120 may be operated to control operation of the output of the local computing devices 115, 120. The output may be a television, liquid crystal display (LCD) monitor, cathode ray tube (CRT) monitor, speaker, tactile output device, and/or the like. In some embodiments, the output may be an integral component of the local computing devices 115, 120. Similarly stated, the output may be directly coupled to the processor. For example, the output may be the integral display of a tablet and/or smartphone. In some embodiments, an output module may include, for example, a High Definition Multimedia Interface™ (HDMI) connector, a Video Graphics Array (VGA) connector, a Universal Serial Bus™ (USB) connector, a tip, ring, sleeve (TRS) connector, and/or any other suitable connector operable to couple the local computing devices 115, 120 to the output.

As described in additional detail herein, at least one of the sensor units 110 may be operable to transmit physiological measurements to the local computing devices 115, 120 and/or to the remote computing device 145 continuously, at scheduled intervals, when requested, and/or when certain conditions are satisfied (e.g., during an at-rest condition). In some embodiments, the rate at which physiological measurements are collected and/or transmitted may increase when the patient is determined to be at-rest.

The remote computing device 145 may be a computing entity operable to enable a remote user to monitor the output of the sensor units 110. The remote computing device 145 may be functionally and/or structurally similar to the local computing devices 115, 120 and may be operable to receive data streams from and/or send signals to at least one of the sensor units 110 via the network 125. The network 125 may be the Internet, an intranet, a personal area network, a local area network (LAN), a wide area network (WAN), a virtual network, a telecommunications network implemented as a wired network and/or wireless network, etc. The remote computing device 145 may receive and/or send signals over the network 125 via communication links 150 and server 135.

The remote computing device 145 may be used by, for example, a health care professional to monitor the output of the sensor units 110. In some embodiments, the remote computing device 145 may receive an indication of physiological measurements when the sensors detect an at-rest condition, when the healthcare provider requests the information, at scheduled intervals, and/or at the request of the healthcare provider and/or the patient 105. For example, the remote computing device 145 may be operable to receive summarized physiological measurements from the server 135 and display the summarized physiological measurements in a convenient format. The remote computing device 145 may be located, for example, at a nurses station or in a patient's room, and may be configured to display a summary of the physiological measurements collected from one or more patients. In some instances, the local computing devices 115, 120 may also be operable to receive and display physiological measurements in much the same way that the remote computing device 145 is operable.

The server 135 may be configured to communicate with the sensor units 110, the local computing devices 115, 120, third-party sensors 130, the remote computing device 145 and databases 140. The server 135 may perform additional processing on signals received from the sensor units 110, local computing devices 115, 120 or third-party sensors 130, or may simply forward the received information to the remote computing device 145 and databases 140. The databases 140 may be examples of electronic health records (“EHRs”) and/or personal health records (“PHRs”), and may be provided by various service providers. The third-party sensor 130 may be a sensor that is not attached to the patient 105 but that still provides data that may be useful in connection with the data provided by sensor units 110. In certain embodiments, the server 135 may be combined with one or more of the local computing devices 115, 120 and/or the remote computing device 145.

The server 135 may be a computing device operable to receive data streams (e.g., from the sensor units 110 and/or the local computing devices 115, 120), store and/or process data, and/or transmit data and/or data summaries (e.g., to the remote computing device 145). For example, the server 135 may receive a stream of heart rate data from a sensor unit 110, a stream of oxygen saturation data from the same or a different sensor unit 110, and a stream of acceleration data from either the same or yet another sensor unit 110. Based on corresponding physiological data and activity data received, the server 135 may be able to determine which physiological measurements were taken during an at-rest period, and may flag the physiological measurements accordingly. In some embodiments, the server 135 may “pull” the data streams, e.g., by querying the sensor units 110 and/or the local computing devices 115, 120. In some embodiments, the data streams may be “pushed” from the sensor units 110 and/or the local computing devices 115, 120 to the server 135. For example, the sensor units 110 and/or the local computing devices 115, 120 may be configured to transmit data as it is generated by or entered into that device. In some instances, the sensor units 110 and/or the local computing devices 115, 120 may periodically transmit data (e.g., as a block of data or as one or more data points).

The server 135 may include a database (e.g., in memory) containing physiological measurements received from the sensor units 110 and/or the local computing devices 115, 120. Additionally, as described in further detail herein, software (e.g., stored in memory) may be executed on a processor of the server 135. Such software (executed on the processor) may be operable to cause the server 135 to monitor, process, summarize, present, and/or send a signal associated with physiological measurements, for example indicating those physiological measurements that were taken during an at-rest period.

Although the server 135 and the remote computing device 145 are shown and described as separate computing devices, in some embodiments, the remote computing device 145 performs the functions of the server 135 such that a separate server 135 may not be necessary. In such an embodiment, the remote computing device 145 receives physiological measurement streams from the sensor units 110 and/or the local computing devices 115, 120, processes the received physiological measurements, and displays the processed physiological measurements as summarized physiological measurements, with at-rest flags indicating those physiological measurements taken during an at-rest period.

Additionally, although the remote computing device 145 and the local computing devices 115, 120 are shown and described as separate computing devices, in some embodiments, the remote computing device 145 performs the functions of the local computing devices 115, 120 such that a separate local computing device 115, 120 may not be necessary. In such an embodiment, the user (e.g., a nurse or a doctor) may manually enter the patient's physiological measurements (e.g., the patient's body temperature) directly into the remote computing device 145.

In the system 100 of FIG. 1, a sensor unit 110 may detect activity data and physiological data of the patient 105. In some embodiments, a single sensor unit 110 may detect both activity data and physiological data. In alternate embodiments, one sensor unit 110 may detect activity data, while a second sensor unit 110 may detect physiological data. In addition, one or sensors may detect physiological measurements of the patient. Activity data received by sensor unit 110 may comprise any one or more of position, velocity, posture, or acceleration data. Physiological data received by the same or a separate sensor unit 110 may comprise any one or more of heart rate, respiration rate, heart rate variability, respiration rate variability, blood pressure, blood oxygen, blood glucose, perspiration, core temperature, electromyography (EMG) data, or electroencephalogram (EEG) data. In addition, one or more sensor unit 110 or third-party sensor 130 may detect environmental data, the environmental data comprising one or more of temperature, humidity, surface pressure, motion or vibration data.

Based on the received activity data and/or physiological data, it may be determined that the patient is at-rest. This determination may be made at the one or more sensor units 110, or may be determined at any one of the local computing devices 115, 120, the remote computing device 145, and/or the server 135. Physiological measurements may be received on an ongoing basis from the one or more sensor units 110. Upon determining that the patient is at-rest, collected physiological measurements may be flagged as having been taken during an at-rest period. This “at-rest flag” may be linked to the physiological measurements transmitted to the patient or clinician to be displayed, for example, at a nurses station or in a patient's room, or alternatively on any of the local computing devices 115, 120 or the remote computing device 145. In some embodiments, the flagged physiological measurements may be displayed on the one or more sensor units 110.

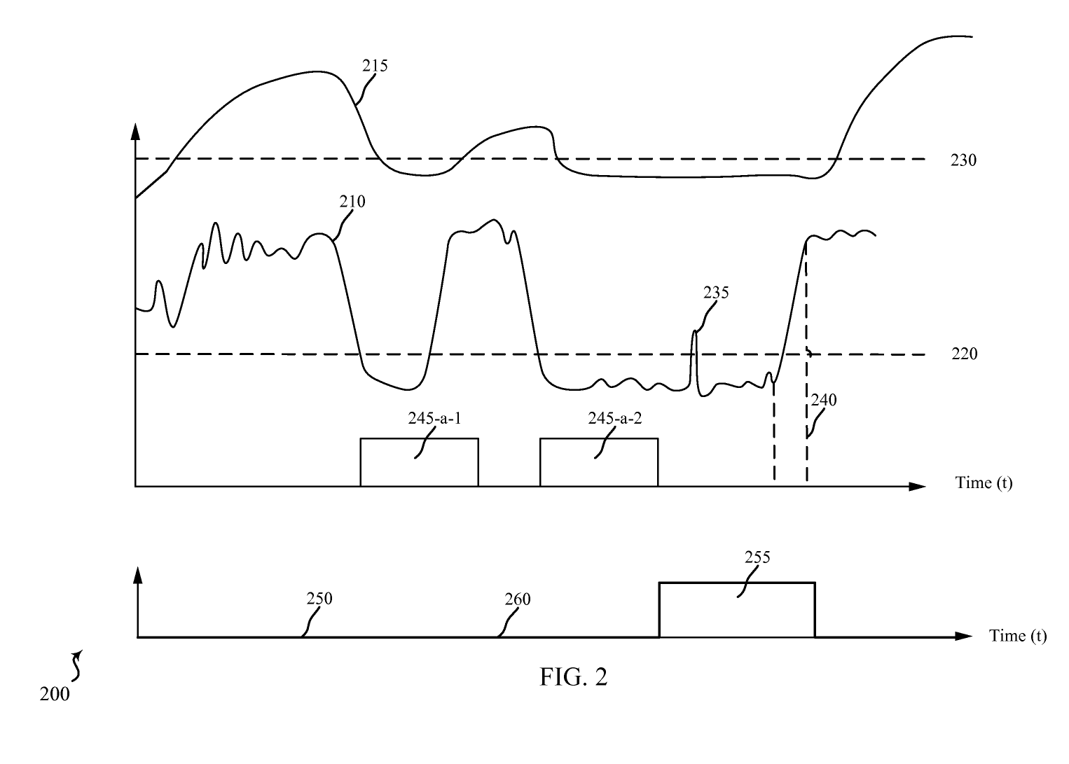

FIG. 2 is a graphical representation 200 of the collecting of physiological data and activity data, and the determination of an at-rest period for a patient based on the collected data. Physiological data and activity data may be collected by sensor units 110, as shown in FIG. 1. In representation 200, collected physiological data is illustrated in the form of a patient physiological level 215, and collected activity data is illustrated in the form of a patient activity level 210. The collected data is collected over a period of time, t.

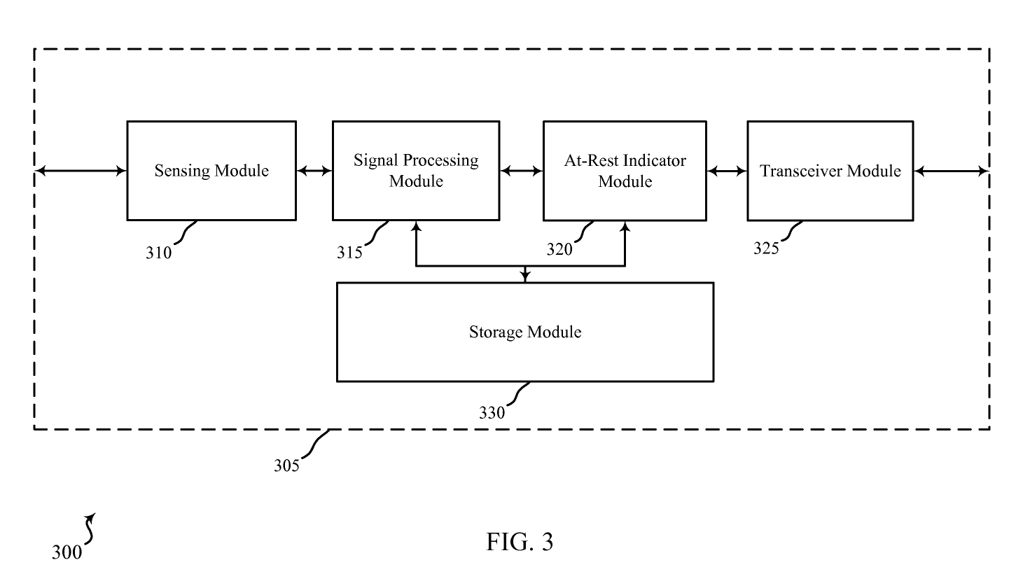

As shown in FIG. 2, patient activity level 210 corresponds to the collected activity data. In one embodiment, a raw acceleration signal (not shown) may be received in the form of acceleration along a single axis, and/or as a magnitude of a multi-dimensional acceleration vector. The raw acceleration signal may then be normalized by the sensor unit 110 or a signal processing module 315 (as described in relation to FIG. 3), the local computing devices 115, 120, the server 135, and/or the remote computing device 145 to provide a patient activity level 210 curve. A patient physiological level 215 may be simultaneously monitored by sensor units 110, for example in the form of the patient's heart rate signal. In alternate embodiments any suitable physiological data, such as respiration rate or blood oxygen saturation, may be monitored.

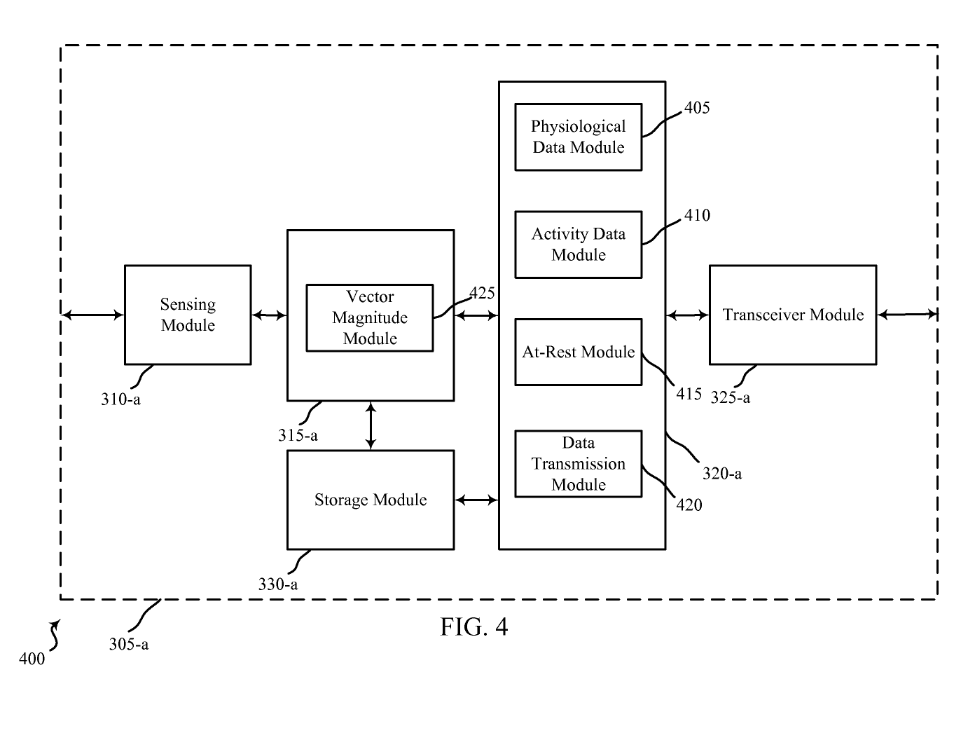

Representation 200 also illustrates a mechanical at-rest threshold 220 and a physiological at-rest threshold 230. A mechanical at-rest threshold 220 may be represented by a metabolic equivalence threshold (MET) of 1 MET, for example. Thus, a determination of whether the patient is mechanically at-rest may be made when the patient activity level 210 is below 1 MET as determined by, for example, an accelerometer in conjunction with a vector magnitude module 425 (as described in relation to FIG. 4).

A physiological at-rest threshold 230 is also shown in representation 200. The physiological at-rest threshold 230 may vary among patients based on individual patient physiological parameters, and may also vary based on the physiological data being monitored. For example, as illustrated in representation 200, the physiological at-rest threshold 230 may represent a heart rate threshold, below which the patient may be determined to be physiologically at-rest. In alternate embodiments, the physiological at-rest threshold may represent a blood oxygen threshold or a perspiration threshold, for example, by which corresponding physiological data may be determined to indicate an at-rest status of a patient.

In one embodiment, a determination of whether a patient is at-rest may be based only on the patient activity level 210 determined by the activity data received; in other words, the patient may be determined to be mechanically at-rest. In other embodiments, a determination of whether a patient is at-rest may be based only on the patient physiological level 215 determined by the physiological data received, meaning that the patient is physiologically at-rest. In still other embodiments, a determination of at-rest status is based on both the patient activity level 210 and the patient physiological level 215. Thus, where the patient physiological level 215 is below the physiological at-rest threshold 230, and where the patient activity level 210 is also below the mechanical at-rest threshold 220, an at-rest flag 250 may be set to “true” 255. During the period in which the at-rest flag 250 is set to “true” 255, monitored physiological measurements may be associated with an at-rest indicator.

In some embodiments, for example where the patient physiological level 215 is indicated by patient heart rate, the physiological at-rest threshold 230 may be selected from any of a predetermined at-rest heart rate; a stable heart rate over a predetermined period of time, such as 30 seconds, 1 minute, 2 minutes or any other suitable period of time; or a heart rate within a range that has historically been associated with the patient being at-rest, such as 100 beats per minute (bpm), 75 bpm, 60 bpm, or any other suitable threshold.

In some embodiments, the at-rest flag 250 is only set to “true” 255 when the patient physiological level 215 and the patient activity level 210 are both below the physiological at-rest threshold 230 and the mechanical at-rest threshold 220, respectively, for a predetermined period of time. The predetermined period of time may be measured by an at-rest timer window 245-a-1, 245-a-2 of any suitable length of time to allow for patient recovery, for example 20 minutes, 10 minutes, 5 minutes, etc. Individual at-rest timer window durations may be adjusted based on individual patients' health and fitness levels. When the at-rest timer has surpassed the at-rest timer window 245-a-1, 245-a-2, the at-rest flag 250 may be set to “true” 255, and the physiological measurements collected thereafter may be associated with an at-rest indicator for the period of time during which the at-rest flag 250 remains set to “true” 255.

As shown in the example illustrated in FIG. 2, the patient activity level 210 and patient physiological level 215 may exceed the mechanical at-rest threshold 220 and physiological at-rest threshold 230, respectively, prior to the completion of the at-rest timer window 245-a-1, such that the at-rest flag 250 may not be set to “true” 255, but instead may remain “false” 260, and the physiological measurements collected may not be associated with an at-rest indicator. However, at a later time, the patient activity level 210 and patient physiological level 215 may remain below the mechanical at-rest threshold 220 and physiological at-rest threshold 230, respectively, beyond completion of the at-rest timer window 245-a-2, such that the at-rest flag 250 may be set to “true” 255 and the physiological measurements collected thereafter may be associated with an at-rest indicator during such time as the patient activity level 210 and patient physiological level 215 remain below the mechanical at-rest threshold 220 and physiological at-rest threshold 230, respectively. In one embodiment, during the time period of transition from an at-rest state to an active state 240, the at-rest flag 250 may remain set to “true” 255 until such time as the patient physiological signal 215 exceeds the physiological at-rest threshold 230. This may occur, for example, when a patient stands up to become mechanically active, but his heart rate does not immediately increase. In other embodiments, the at-rest flag 250 may be set to “false” 260 if either or both of the mechanical at-rest threshold 220 or physiological at-rest threshold 230 are surpassed, such that the physiological measurements collected are no longer associated with an at-rest indicator.

FIG. 3 shows a block diagram 300 that includes apparatus 305, which may be an example of one or more aspects of the local computing devices 115, 120 and/or remote computing device 145, or may alternatively be an example of one or more aspects of the one or more sensor units 110 (of FIG. 1), for use in physiological measurement monitoring, in accordance with various aspects of the present disclosure. In some examples, the apparatus 305 may include a sensing module 310, a signal processing module 315, an at-rest indicator module 320, a transceiver module 325, and a storage module 330. Each of these components may be in communication with each other.

The components of the apparatus 305 may, individually or collectively, be implemented using one or more application-specific integrated circuits (ASICs) adapted to perform some or all of the applicable functions in hardware. Alternatively, the functions may be performed by one or more other processing units (or cores), on one or more integrated circuits. In other examples, other types of integrated circuits may be used (e.g., Structured/Platform ASICs, Field Programmable Gate Arrays (FPGAs), and other Semi-Custom ICs), which may be programmed in any manner known in the art. The functions of each unit may also be implemented, in whole or in part, with instructions embodied in a memory, formatted to be executed by one or more general or application-specific processors.

The at-rest indicator module 320 may be configured to monitor the physiological data and activity data sensed by the sensing module 310 and processed by the signal processing module 315, described in more detail below with respect to FIG. 4. In some examples, the at-rest indicator module 320 may determine physiological at-rest and mechanical at-rest threshold levels, where the determined threshold levels may be based, at least in part, on the monitored physiological data and activity data. The at-rest indicator module 320 may determine that an at-rest status indicator should be triggered, based on at least one of the monitored physiological data and activity data, and the respective determined thresholds.

In some examples where apparatus 305 is part of one or more of the local computing devices 115, 120 or remote computing device 145, the transceiver module 325 may be operable to receive data streams from the one or more sensor units 110, as well as to send and/or receive other signals between the sensor units 110 and either the local computing devices 115, 120 or the remote computing device 145 via the network 125 and server 135. In one embodiment, the transceiver module 325 may receive data streams from the sensor units 110 and also forward the data streams to other devices. The transceiver module 325 may include wired and/or wireless connectors. For example, in some embodiments, sensor units 110 may be portions of a wired or wireless sensor network, and may communicate with the local computing devices 115, 120 and/or remote computing device 145 using either a wired or wireless network. The transceiver module 325 may be a wireless network interface controller (“NIC”), Bluetooth® controller, IR communication controller, ZigBee 0 controller and/or the like.

In some embodiments, where apparatus 305 is part of one or more of the sensor units 110, the transceiver module 325 may send a signal to a local computing device 115, 120 and/or remote computing device 145 if the patient taps the device, as detected by the one or more sensor units 110. In some embodiments, the patient may tap the sensor unit 110 such that communication of the signal occurs directly from the apparatus 305. In other embodiments, the patient may tap the sensor unit 110, which may send a signal to apparatus 305, wherein apparatus 305 may be part of one or more of the local computing devices 115, 120 or remote computing device 145. Alternatively, the transceiver module 325 may send a signal if an acceleration-based alert signal is detected by the sensor unit 110, as illustrated and described in commonly owned U.S. Patent Application No. 61/882,268, filed Sep. 25, 2013, which is incorporated herein by reference in its entirety.

The local computing device 115, 120 and/or remote computing device 145, upon receiving a signal from the transceiver module 325, may send alerts using such methods as short message service (SMS) text messages, email, or any other suitable means. In embodiments where apparatus 305 is part of one or more of the sensor units 110, transceiver module 325 may send a signal to the local computing device 115, 120 and/or remote computing device 145. In alternative embodiments, where apparatus 305 is part of one or more of the local computing device 115, 120 or remote computing device 145, the transceiver module 325 may communicate the signal within the apparatus 305. For example, if the signal indicates that a vital sign exceeds a threshold, the monitoring station may send information to the patient, a clinician, support personnel, a family member, etc. The information may include web content, educational information, or support information, or a request to take part in an activity, such as a fitness test, questionnaire, or exercise program. The information may further request that the patient make a dietary or sleeping pattern change. In some embodiments, the local computing device 115, 120 and/or remote computing device 145 may book an appointment for the patient with a caregiver.

In some embodiments, where apparatus 305 is part of one or more of the sensor units 110, transceiver module 325 may be operable to determine when a local computing device 115, 120 and/or remote computing device 145 is available to receive a signal from the transceiver module 325. For example, the transceiver module 325 may detect when a local computing device 115, 120 and/or remote computing device 145 is within a certain distance of the apparatus 305. In such an embodiment, the transceiver module 325 may push data to the local computing device 115, 120 and/or remote computing device 145. In other embodiments, physiological data may be pulled from the transceiver module 325 by the local computing device 115, 120 and/or remote computing device 145. In other words, the transceiver module 325 may receive a signal requesting physiological measurements from the local computing device 115, 120 and/or remote computing device 145.

In some examples, where apparatus 305 is part of one or more of the local computing device 115, 120 or remote computing device 145, or alternatively where apparatus 305 is part of one or more of the sensor units 110, the signal processing module 315 may include circuitry, logic, hardware and/or software for processing the data streams received from the sensor units 110. The signal processing module 315 may include filters, analog-to-digital converters and other digital signal processing units. Data processed by the signal processing module 315 may be stored in a buffer, for example, in the storage module 330. The storage module 330 may include magnetic, optical or solid-state memory options for storing data processed by the signal processing module 315. The at-rest indicator module 320 may access the data stored in the storage module 330 and output an at-rest indicator associated with the physiological measurements.

FIG. 4 shows a block diagram 400 that includes apparatus 305-a, which may be an example of apparatus 305 (as illustrated in FIG. 3), in accordance with various aspects of the present disclosure. In some embodiments, apparatus 305-a may be part of one or more of the local computing device 115, 120 or remote computing device 145; in alternate embodiments, apparatus 305-a may be part of one or more of the sensor units 110. In some examples, the apparatus 305-a may include a sensing module 310-a, a signal processing module 315-a, an at-rest indicator module 320-a, a transceiver module 325-a, and a storage module 330-a, which may be examples of the sensing module 310, signal processing module 315, at-rest indicator module 320, transceiver module 325, and storage module 330 of FIG. 3, respectively. In some examples, the signal processing module 315-a may also include a vector magnitude module 425. The vector magnitude module 425 may be used in aspects of correlating acceleration data received from one or more sensor units 110 with activity data of the patient. In some examples, the at-rest indicator module 320-a may include a physiological data module 405, an activity data module 410, an at-rest module 415, and a data transmission module 420. The modules 405, 410, 415 and/or 420 may each be used in aspects of collecting activity data and physiological data and using the data to associate collected physiological measurements with an at-rest period. Additionally, while FIG. 4 illustrates a specific example, the functions performed by each of the modules 405, 410, 415 and/or 420 may be combined or implemented in one or more other modules.

The vector magnitude module 425 may further comprise an analog to digital converter (ADC, not shown). In one embodiment, at least one of the sensor units 110 may comprise an accelerometer. The accelerometer may be operable to send a signal associated with any one of detected position, velocity, and/or acceleration of the sensor unit 110 to the ADC, which may sample the analog signal output by the accelerometer and convert the analog signal into a digital acceleration signal. The ADC may then send the digital acceleration signal to the vector magnitude module 425, which may be operable to correlate the digital acceleration signal to an activity level of the patient. In some embodiments, the vector magnitude module 425 may calculate a metabolic equivalency (expressed in METs) of the patient's activity. In some embodiments, the vector magnitude module 425 may be customizable based on the patient's age, gender, body weight, etc. The metabolic equivalency output by the vector magnitude module 425 may be used to determine the mechanical at-rest status of the patient. For example, by convention, 1 MET is the at-rest metabolic rate of an average individual, and therefore a patient may be determined to be mechanically at-rest when the vector magnitude module 425 calculates a metabolic equivalency of 1 MET based on the digital acceleration signal received from the ADC. Although described as separate from the one or more sensor units 110, in some embodiments apparatus 305-a may be a component of one or more sensor units 110 such that the signal from the accelerometer is directed within the apparatus 305-a component of the one or more sensor units 110 to convert the analog signal to a digital acceleration signal.

The physiological data module 405 may be used to receive processed physiological data signals received from signal processing module 315-a to determine whether the physiological data is above or below the physiological at-rest threshold 230 (of FIG. 2). As discussed above with respect to FIG. 2, the physiological at-rest threshold may comprise a predetermined at-rest physiological reading, a stable physiological reading over a predetermined period of time, or a physiological reading within a range that has historically been associated with the patient being at-rest.

The activity data module 410 may similarly be used to receive processed activity data signals received from signal processing module 315-a to determine whether the activity data is above or below the mechanical at-rest threshold 220 (of FIG. 2). As discussed above with respect to FIG. 2, the mechanical at-rest threshold 220 may comprise a metabolic equivalence threshold of 1 MET, wherein in some embodiments an accelerometer is used to determine whether the patient activity level 210 is below 1 MET.

The at-rest module 415 may be used to determine, based on at least one of the determinations of physiological data module 405 and activity data module 410, whether the patient is physiologically at-rest, mechanically at-rest, or both. Upon determination that the patient is at-rest, the data transmission module 420 will associate an at-rest indicator with one or more physiological measurements, such that the physiological measurements transmitted to the caregiver or patient, or alternatively displayed on the one or more sensor units 110, are flagged as having been taken during periods of at-rest status.

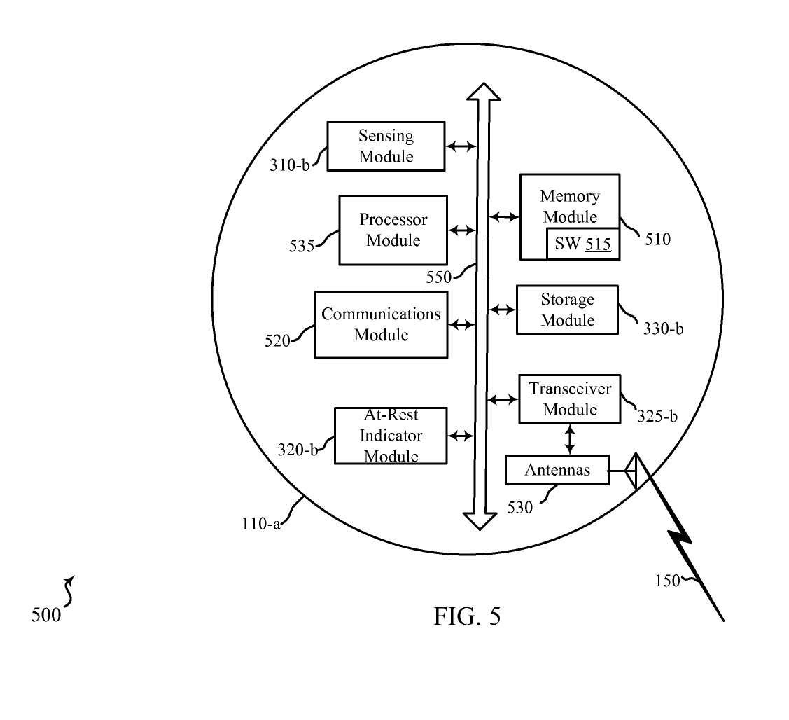

FIG. 5 shows a block diagram 500 of a sensor unit 110-a for use in remote physiological monitoring, in accordance with various aspects of the present disclosure. The sensor unit 110-a may have various configurations. The sensor unit 110-a may, in some examples, have an internal power supply (not shown), such as a small battery, to facilitate mobile operation. In some examples, the sensor unit 110-a may be an example of one or more aspects of one of the sensor units 110 and/or apparatus 305 described with reference to FIGS. 1 and/or 3. The sensor unit 110-a may be configured to implement at least some of the features and functions described with reference to FIGS. 1, 3 and/or 4.

The sensor unit 110-a, which may include one or more aspects of apparatus 305 (as described in FIGS. 3 and/or 4) may include a sensing module 310-b, a processor module 535, a memory module 510, a communications module 520, at least one transceiver module 325-a, at least one antenna (represented by antennas 530), a storage module 330-b, and/or an at-rest indicator module 320-b. Each of these components may be in communication with each other, directly or indirectly, over one or more buses 550. The sensing module 310-b, the at-rest indicator module 320-b, the transceiver module 325-b, and the storage module 330-b may be examples of the sensing module 310, the at-rest indicator module 320, the transceiver module 325, and the storage module 330, respectively, of FIG. 3.

The memory module 510 may include random access memory (RAM) or read-only memory (ROM). The memory module 510 may store computer-readable, computer-executable software (SW) code 515 containing instructions that are configured to, when executed, cause the processor module 535 to perform various functions described herein for communicating, for example, at-rest status. Alternatively, the software code 515 may not be directly executable by the processor module 535, but may be configured to cause the sensor unit 110-a (e.g., when compiled and executed) to perform various of the functions described herein.

The processor module 535 may include an intelligent hardware device, e.g., a CPU, a microcontroller, an ASIC, etc. The processor module 535 may process information received through the transceiver module 325-b or information to be sent to the transceiver module 325-b for transmission through the antenna 530. The processor module 535 may handle, alone or in connection with the at-rest indicator module 320-b, various aspects of signal processing as well as determining and transmitting at-rest status indicators.

The transceiver module 325-b may include a modem configured to modulate packets and provide the modulated packets to the antennas 530 for transmission, and to demodulate packets received from the antennas 530. The transceiver module 325-b may, in some examples, be implemented as one or more transmitter modules and one or more separate receiver modules. The transceiver module 325-b may support at-rest status-related communications. The transceiver module 325-b may be configured to communicate bi-directionally, via the antennas 530 and communication link 150, with, for example, local computing devices 115, 120 and/or the remote computing device 145 (via network 125 and server 135 of FIG. 1). Communications through the transceiver module 325-b may be coordinated, at least in part, by the communications module 520. While the sensor unit 110-a may include a single antenna, there may be embodiments in which the sensor unit 110-a may include multiple antennas 530.

The at-rest indicator module 320-b may be configured to perform or control some or all of the features or functions described with reference to FIGS. 1, 2, 3 and/or 4 related to at-rest status indicator generation and transmission. For example, the at-rest indicator module 320-b may be configured to monitor the physiological data and activity data sensed by the sensing module 310-b. In some examples, the at-rest indicator module 320-b may determine physiological at-rest and mechanical at-rest threshold levels, where the determined threshold levels may be based, at least in part, on the monitored physiological data and activity data. The at-rest indicator module 320-b may determine that an at-rest status indicator should be triggered, based on at least one of the monitored physiological data and activity data, and the respective determined thresholds. The at-rest status indicators and associated physiological measurements (both the data, either processed or unprocessed, to which the at-rest status indicator pertains as well as contextual data) may be transmitted to either a local computing device 115, 120 or a remote computing device 145. The at-rest indicator module 320-b, or portions of it, may include a processor, or some or all of the functions of the at-rest indicator module 320-b may be performed by the processor module 535 or in connection with the processor module 535. Additionally, the at-rest indicator module 320-b, or portions of it, may include a memory, or some or all of the functions of the at-rest indicator module 320-b may use the memory module 510 or be used in connection with the memory module 510.

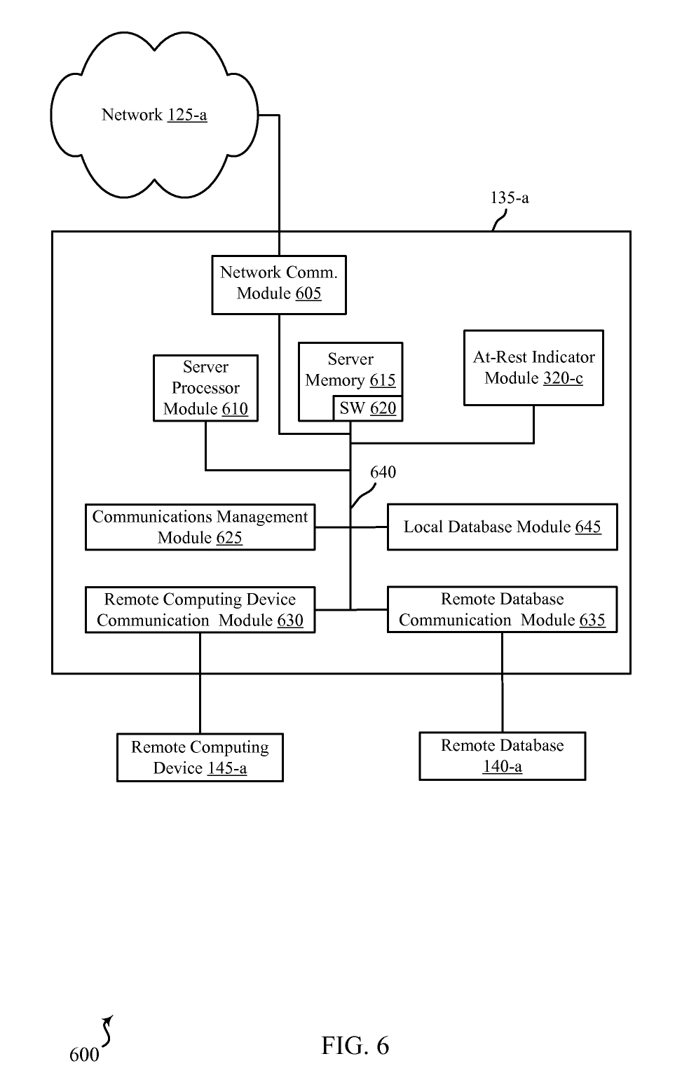

FIG. 6 shows a block diagram 600 of a server 135-a for use in determining at-rest status of a patient, in accordance with various aspects of the present disclosure. In some examples, the server 135-a may be an example of aspects of the server 135 described with reference to FIG. 1. In other examples, the server 135-a may be implemented in either the local computing devices 115, 120 or the remote computing device 145 of FIG. 1. The server 135-a may be configured to implement or facilitate at least some of the features and functions described with reference to the server 135, the local computing devices 115, 120 and/or the remote computing device 145 of FIG. 1.

The server 135-a may include a server processor module 610, a server memory module 615, a local database module 645, and/or a communications management module 625. The server 135-a may also include one or more of a network communication module 605, a remote computing device communication module 630, and/or a remote database communication module 635. Each of these components may be in communication with each other, directly or indirectly, over one or more buses 640.

The server memory module 615 may include RAM and/or ROM. The server memory module 615 may store computer-readable, computer-executable code 620 containing instructions that are configured to, when executed, cause the server processor module 610 to perform various functions described herein related to presenting at-rest indicated physiological measurements. Alternatively, the code 620 may not be directly executable by the server processor module 610 but may be configured to cause the server 135-a (e.g., when compiled and executed) to perform various of the functions described herein.

The server processor module 610 may include an intelligent hardware device, e.g., a central processing unit (CPU), a microcontroller, an ASIC, etc. The server processor module 610 may process information received through the one or more communication modules 605, 630, 635. The server processor module 610 may also process information to be sent to the one or more communication modules 605, 630, 635 for transmission. Communications received at or transmitted from the network communication module 605 may be received from or transmitted to sensor units 110, local computing devices 115, 120, or third-party sensors 130 via network 125-a, which may be an example of the network 125 described in relation to FIG. 1. Communications received at or transmitted from the remote computing device communication module 630 may be received from or transmitted to remote computing device 145-a, which may be an example of the remote computing device 145 described in relation to FIG. 1. Communications received at or transmitted from the remote database communication module 635 may be received from or transmitted to remote database 140-a, which may be an example of the remote database 140 described in relation to FIG. 1. Additionally, a local database may be accessed and stored at the server 135-a. The local database module 645 may be used to access and manage the local database, which may include data received from the sensor units 110, the local computing devices 115, 120, the remote computing devices 145 or the third-party sensors 130 (of FIG. 1).

The server 135-a may also include an at-rest indicator module 320-c, which may be an example of the at-rest indicator module 320 of apparatus 305 described in relation to FIGS. 3, 4 and/or 5. The at-rest indicator module 320-c may perform some or all of the features and functions described in relation to the at-rest indicator module 320, including selecting and obtaining from either the local database module 645 or the remote database 140-a data corresponding to the physiological data and activity data, determining whether the physiological data and/or the activity data falls below the physiological at-rest threshold and/or the mechanical at-rest threshold, respectively, determining on the basis of this determination whether the patient is at-rest, and associating an at-rest indicator with one or more physiological measurements of the patient collected by the one or more sensor units 110.

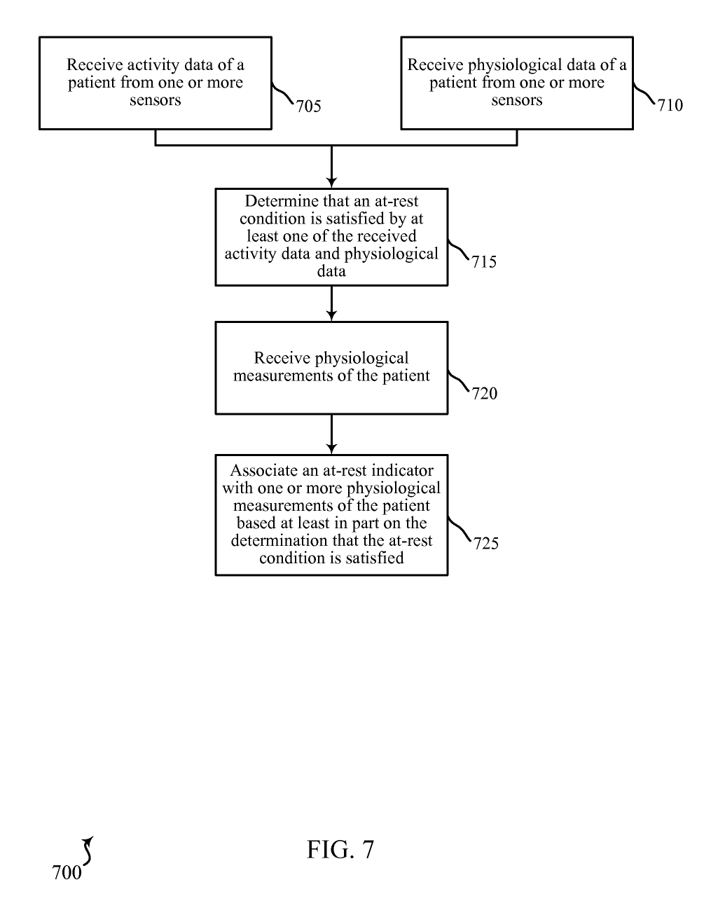

FIG. 7 is a flow chart illustrating an example of a method 700 of remote physiological monitoring, in accordance with various aspects of the present disclosure. For clarity, the method 700 is described below with reference to aspects of one or more of the local computing devices 115, 120, remote computing device 145, and/or server 135 described with reference to FIGS. 1, and/or 6, or aspects of one or more of the apparatus 305 described with reference to FIGS. 3 and/or 4. In some examples, a local computing device, remote computing device or server such as one of the local computing devices 115, 120, remote computing device 145, server 135 and/or an apparatus such as one of the apparatuses 305 may execute one or more sets of codes to control the functional elements of the local computing device, remote computing device, server or apparatus to perform the functions described below. In alternate embodiments, method 700 may be carried out at one or more sensor units 110.

At block 705, the method 700 may include receiving activity data of a patient from one or more sensors. As discussed above, activity data may comprise acceleration data, position data, posture data, etc.

At block 710, the method 700 may include receiving physiological data of a patient from one or more sensors. The one or more sensors for receiving physiological data may be the same one or more sensors for receiving activity data, or may be separate sensors. As discussed above, physiological data may comprise heart rate, respiration rate, blood oxygen levels, etc.

At block 715, the method 700 may include determining that an at-rest condition is satisfied by at least one of the received activity data and physiological data. As described above, it may be determined that an at-rest condition is satisfied by determining whether the received activity data and/or the received physiological data falls below the mechanical at-rest threshold and/or the physiological at-rest threshold, respectively.

At block 720, the method 700 may include receiving physiological measurements of the patient. As discussed above, physiological measurements may be collected on an ongoing basis, or may be collected at discrete time intervals based, for example, on patient or caregiver request, or based on a predetermined schedule.

At block 725, the method 700 may include associating an at-rest indicator with one or more physiological measurements of the patient based at least in part on the determination that an at-rest condition is satisfied. Thus, while physiological measurements may be collected and/or transmitted on an ongoing basis, only those physiological measurements collected during an at-rest period will be associated with an at-rest indicator. The caregiver may then base diagnoses on the received physiological measurements with the accompanying background information of whether the patient was at-rest when the subject physiological measurements were observed.

In some embodiments, the operations at blocks 705, 710, 715, 720 or 725 may be performed using the at-rest indicator module 320 described with reference to FIGS. 3, 4 and/or 5. Nevertheless, it should be noted that the method 700 is just one implementation and that the operations of the method 700 may be rearranged or otherwise modified such that other implementations are possible.

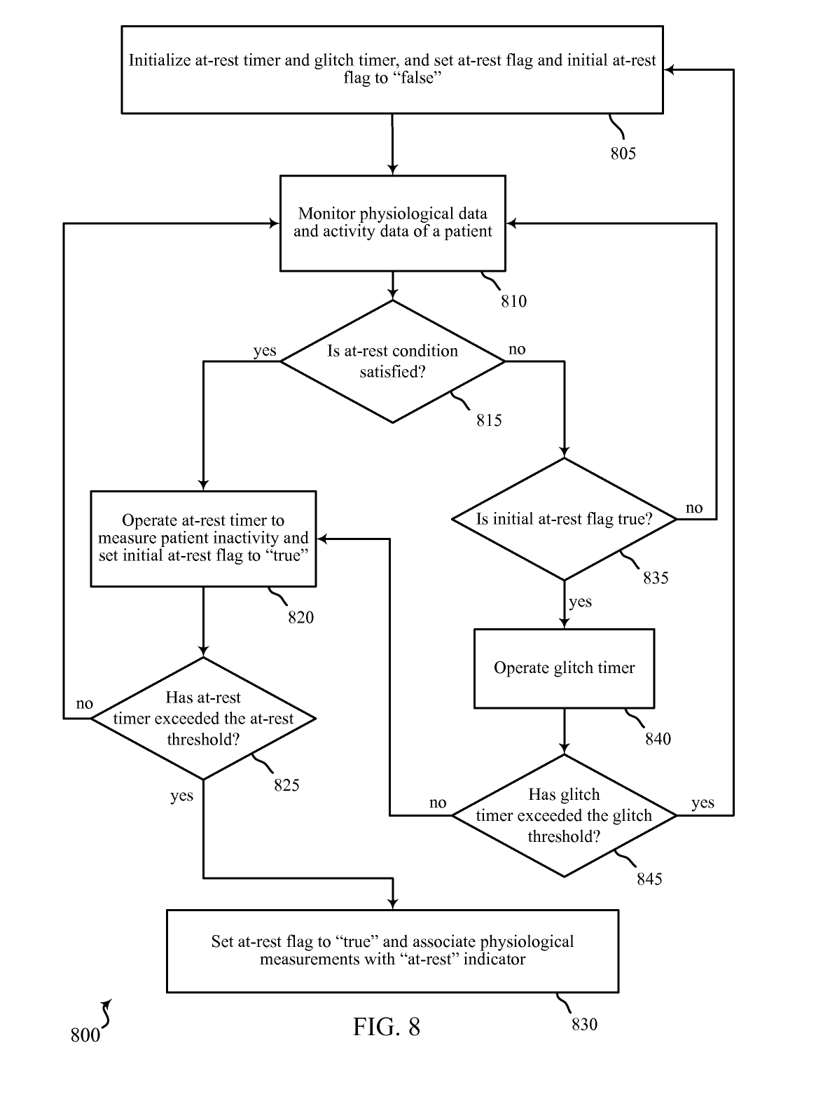

FIG. 8 is a flow chart illustrating an example of a method 800 for determining whether an at-rest condition is met on the basis of the collecting of physiological and activity data, as well as the use of at-rest timers, in accordance with various aspects of the present disclosure. For clarity, the method 800 is described below with reference to aspects of one or more of the local computing devices 115, 120, remote computing device 145, and/or server 135 described with reference to FIGS. 1 and/or 6, or aspects of one or more of the apparatus 305 described with reference to FIGS. 3 and/or 4. In alternative embodiments, method 800 could be performed at one or more of the sensor units 110. In some examples, a sensor unit, a local computing device, remote computing device or server such as one of the sensor units 110, local computing devices 115, 120, remote computing device 145, server 135 (of FIGS. 1, 5 and/or 6, for example) and/or an apparatus such as one of the apparatuses 305 (of FIGS. 3 and/or 4) may execute one or more sets of codes to control the functional elements of the local computing device, remote computing device, server or apparatus to perform the functions described below.

The method 800 may be used, for example, to monitor one or more of a patient's physiological measurements and to present the associated data to a healthcare professional (e.g., a nurse or doctor) with associated at-rest indicators to make clear to the caregiver which physiological measurements were collected while the patient was at-rest. In some embodiments, the patient may be monitored in a hospital, a hospice or other healthcare related facility. In other embodiments, the patient may be monitored at home and the patient's physiological measurements may be streamed to the location of the healthcare provider. The vital signs or other physiological measurements being monitored may include, but are not limited to, the patient's heart rate, heart rate variability, respiratory rate, respiratory rate variability, body temperature, blood pressure, blood oxygen saturation, EMG data, ECG data, weight, blood sugar, and/or the like.

As shown in FIG. 8, at step 805, the method 800 includes initializing an at-rest timer and a glitch timer, and setting an at-rest flag and an initial at-rest flag to “false.” As described above with reference to FIG. 2, in some embodiments, the at-rest flag 250 is only set to “true” 255 when the patient physiological level 215 and the patient activity level 210 are both below the physiological at-rest threshold 230 and the mechanical at-rest threshold 220, respectively, for a predetermined period of time. The predetermined period of time may be measured by an at-rest timer window 245-a-1, 245-a-2 of any suitable length to allow for patient recovery, for example 20 minutes, 10 minutes, 5 minutes, etc. Individual at-rest timer window durations may be adjusted based on individual patients' health and fitness levels. The initial at-rest flag and glitch timer will be discussed in detail below.

At step 810, the method 800 includes monitoring physiological data and activity data of a patient. For example, the local computing devices 115, 120, remote computing device 145 and/or server 135 shown and described above with reference to FIG. 1 may receive one or more physiological data and activity data streams from the sensor units 110, third-party sensors 130, and/or the local computing devices 115, 120 and may store the received data, for example, in a database. Each of the received data streams may be associated with a different physiological or activity parameter and may be received from one or more sensor units 110. For example, the server 135 may receive a stream of a patient's velocity from an accelerometer sensor 110 worn by the patient, and a stream of the patient's heart rate data that was manually entered at the local computing devices 115, 120. Alternatively, physiological data and activity data may be received from the same sensor unit 110. In some embodiments, physiological data and activity data collected by the one or more sensor units 110 may remain at the one or more sensor units 110 for local processing, without the use of local computing devices 115, 120 or remote computing device 145.

At step 815, the method 800 includes determining whether an at-rest condition is satisfied. As described above with respect to FIG. 2, determining whether an at-rest condition is satisfied may comprise determining whether the patient is mechanically at-rest and/or is physiologically at-rest. For example, in one embodiment, a determination of whether a patient is at-rest may be based only on the patient activity level 210 determined by the activity data received; in other words, the patient may be determined to be mechanically at-rest. In other embodiments, a determination of whether a patient is at-rest may be based only on the patient physiological level 215 determined by the physiological data received, meaning that the patient is physiologically at-rest. In still other embodiments, a determination of at-rest status is based on both the patient activity level 210 and the patient physiological level 215, the latter shown in FIG. 2 as a heart rate signal. Thus, where the patient physiological level 215 is below the physiological at-rest threshold 230, and where the patient activity level 210 is also below the mechanical at-rest threshold 220, the at-rest condition may be determined to be satisfied.

In some embodiments, the patient may be determined to be physiologically at-rest based upon reaching a predetermined physiological benchmark or benchmark range, for example a heart rate of 75-85 bpm, or may be determined to be physiologically at-rest based upon maintaining a stable physiological parameter over a predetermined period of time. Additionally, in some embodiments the patient may be determined to be mechanically at-rest when the patient activity level is below a predetermined mechanical at-rest threshold. In one embodiment, the mechanical at-rest threshold may be represented by a metabolic equivalence threshold of 1 MET as determined by, for example, an accelerometer in conjunction with a vector magnitude module 425, as described with reference to FIG. 4.

In the instance that the at-rest condition is determined to be satisfied at step 815 on the basis of the monitored physiological data, the monitored activity data, or both, the method 800 may include operating an at-rest timer to measure patient inactivity and setting an initial at-rest flag to “true,” as shown in step 820. The at-rest timer may be operated for a predetermined period of time to determine whether the patient has had sufficient time to recover from any previous physical activity. For example, where a patient has recently run up a flight of stairs and has now come to a stop, although the patient is no longer mechanically active, the patient may require, for example, 5-10 minutes, depending on individual health and fitness levels, to physiologically recover from the exertion and to reach a physiologically at-rest state.

In some embodiments, a caregiver chooses the length of recovery for the at-rest timer. For example, the caregiver, using the local computing devices 115, 120 or remote computing device 145, may determine and enter the length of time for which it may take the individual patient to physiologically recover from exertion, based on the patient's individual health and fitness levels. In other embodiments, the length of recovery may be preselected. For example, software executing on the processor of the local computing devices 115, 120, the remote computing device 145, and/or the server 135 may preselect the recovery length. In still other embodiments, the length of recovery may be inputted into the one or more sensor units 110 directly.

The initial at-rest flag may be set to “true” at step 820 to indicate that the patient has initially satisfied the at-rest condition. This initial at-rest condition may be used in conjunction with operation of a glitch timer, as discussed in further detail below.

At step 825, the method 800 includes determining whether the at-rest timer has exceeded the predetermined at-rest threshold. As discussed above, the duration or window of the at-rest timer may be tailored to suit individual patients' health and fitness needs. For example, an elderly patient or a patient having a high body mass index may require a longer at-rest timer duration than a younger patient or a patient having a lower body mass index, the latter of which patients may require shorter periods of time to recover from exertion. As shown in FIG. 2, if the at-rest timer window 245-a-2 has exceeded the predetermined at-rest threshold and the patient has remained in an at-rest state, the at-rest flag will be set to “true,” and physiological measurements will be associated with an at-rest indicator, as shown in step 830 of FIG. 8. The at-rest flagged physiological measurements may then be transmitted to the caregiver, or to a local or remote computing device 115, 120 or 145, such that the caregiver may then be able to determine for which intervals of time received physiological measurements were recorded when the patient was at-rest. In alternate embodiments, the at-rest flagged physiological measurements may be displayed directly on the one or more sensor units 110. This will assist the caregiver in determining whether observed elevated blood pressure, heart rate, respiratory rate, etc. was due to physical exertion (when the patient was not at-rest), or possibly due to some underlying health issue (when the patient was at-rest).

If, at step 825, it is determined that the at-rest timer has not exceeded the at-rest threshold, method 800 will return to step 810 to continue to monitor physiological data and activity data of the patient. As the at-rest timer continues to operate, the physiological data and activity data will be constantly monitored, or in some embodiments monitored at preselected intervals, to determine whether the at-rest condition continues to be satisfied, as shown in step 815. If the at-rest condition continues to be satisfied by the monitored activity data and/or physiological data, and the at-rest timer proceeds to meet or exceed the predetermined at-rest threshold at step 825, the at-rest flag will be set to “true” and physiological measurements will be associated with an at-rest indicator at step 830, after which the flagged physiological measurements can be transmitted to a computing device or caregiver, or alternatively displayed on the one or more sensor units 110.

In the alternative, if, as the at-rest timer continues to operate, and the physiological data and/or activity data monitored at some point fail to satisfy the at-rest condition at step 815, at step 835 the method 800 will include determining whether an initial at-rest flag is true. As discussed above, if the at-rest condition was initially determined to be satisfied at step 815, then the at-rest timer was operated at step 820 and the initial at-rest flag was set to “true.” Thus, the determination at step 835 is affirmative, that the initial at-rest flag is true. This scenario may result because, for example, the patient has been seated and relaxed such that he is determined to be physiologically and mechanically at-rest, but has at some point shifted to a different position, for example onto his back, or has raised his arm to scratch his head, or any number of other minor movements. This transient movement may thus temporarily cause at least the activity data, and possibly the physiological data, to spike above the mechanical at-rest threshold and/or the physiological at-rest threshold, respectively, as shown by spike 235 in FIG. 2, such that the at-rest condition is no longer satisfied. In order to determine whether this movement represents a minor movement (a “glitch”) or is instead indicative of the patient becoming active, a glitch timer is operated at step 840 of method 800. At step 845, it may be determined whether the glitch timer has exceeded a predetermined glitch threshold. The glitch threshold may correspond to a length of time longer than a typical transitory movement, but shorter than the lag time between when a patient begins to exert himself and when the patient's vital signs become elevated. For example, the glitch threshold may correspond to 30 seconds, 45 seconds, 1 minute, or any other suitable time period. Various glitch timer durations may be selected based on individual patient parameters. If the spike in patient physiological or mechanical activity exceeds the glitch threshold, then it may be determined that the patient has not made a transitory movement, but has instead become active again such that the patient is no longer at-rest. In this instance, the method 800 will return to step 805, where the at-rest timer and glitch timer will be initialized, and the at-rest flag and initial at-rest flag will be set to “false.” If, in the alternative, the spike in patient physiological and/or mechanical activity does not exceed the glitch threshold, the method 800 will return to step 820, where the at-rest timer will be operated to measure patient inactivity. In this instance, the initial at-rest flag has already been set to “true,” such that no further action is needed in this regard. Once the at-rest timer has met or exceeded the at-rest threshold, the at-rest flag will be set to “true” and physiological measurements will be associated with indications that the measurements were taken while the patient was at-rest, as shown in step 830.

In an alternate embodiment not shown in the method 800 of FIG. 8, but illustrated in FIG. 2, the at-rest flag 250 may have already been set to “true” 255 and the physiological measurements already transmitted with an indication of having been taken at-rest, when the patient may make a transitory movement, represented by spike 235 in FIG. 2. At this point, the glitch timer may be operated simultaneously to the transmission of physiological measurements, and if the glitch timer does not exceed the glitch threshold, the physiological measurements may continue to be transmitted with an indication of at-rest status until such time as the at-rest condition is no longer satisfied. In this way, the method may avoid interrupting the transmission of at-rest physiological measurements on the basis of transient patient movements that have no effect on the patient's at-rest status. If, on the other hand, the glitch timer does exceed the glitch threshold, the at-rest condition will no longer be satisfied and the physiological measurements will no longer be associated with an at-rest indicator.

Referring again to FIG. 8, if, in an alternate scenario, after initializing an at-rest timer and a glitch timer, and setting an at-rest flag and an initial at-rest flag to “false” at step 805, physiological data and activity data of the patient has been monitored at step 810 and it has been determined at step 815 that the at-rest condition is not satisfied, at step 835 it will be determined whether the initial at-rest flag is true. In this instance, because the at-rest condition has not yet been initially satisfied, and therefore the initial at-rest flag has not been set to “true” at step 820, the method 800 will return to step 810 to continue to monitor physiological data and activity data of the patient.

The above description provides examples, and is not limiting of the scope, applicability, or configuration set forth in the claims. Changes may be made in the function and arrangement of elements discussed without departing from the spirit and scope of the disclosure. Various embodiments may omit, substitute, or add various procedures or components as appropriate. For instance, the methods described may be performed in an order different from that described, and various steps may be added, omitted, or combined. Also, features described with respect to certain embodiments may be combined in other embodiments.

The detailed description set forth above in connection with the appended drawings describes exemplary embodiments and does not represent the only embodiments that may be implemented or that are within the scope of the claims. The term “exemplary” used throughout this description means “serving as an example, instance, or illustration,” and not “preferred” or “advantageous over other embodiments.” The detailed description includes specific details for the purpose of providing an understanding of the described techniques. These techniques, however, may be practiced without these specific details. In some instances, well-known structures and devices are shown in block diagram form in order to avoid obscuring the concepts of the described embodiments.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, multiple microprocessors, one or more microprocessors in conjunction with a DSP core, or any other such configuration. A processor may in some cases be in electronic communication with a memory, where the memory stores instructions that are executable by the processor.

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope and spirit of the disclosure and appended claims. For example, due to the nature of software, functions described above may be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items indicates a disjunctive list such that, for example, a list of “at least one of A, B, or C” means A or B or C or AB or AC or BC or ABC (i.e., A and B and C).

A computer program product or computer-readable medium both include a computer-readable storage medium and communication medium, including any mediums that facilitate transfer of a computer program from one place to another. A storage medium may be any medium that may be accessed by a general purpose or special purpose computer. By way of example, and not limitation, a computer-readable medium may comprise RAM, ROM, EEPROM, CD-ROM or other optical disk storage, magnetic disk storage or other magnetic storage devices, or any other medium that may be used to carry or store desired computer-readable program code in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote light source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include compact disc (CD), laser disc, optical disc, digital versatile disc (DVD), floppy disk and Blu-ray disc where disks usually reproduce data magnetically, while discs generally reproduce data optically with lasers. Combinations of the above are also included within the scope of computer-readable media.

The previous description of the disclosure is provided to enable a person skilled in the art to make or use the disclosure. Various modifications to the disclosure will be readily apparent to those skilled in the art, and the generic principles defined herein may be applied to other variations without departing from the spirit or scope of the disclosure. Throughout this disclosure the term “example” or “exemplary” indicates an example or instance and does not imply or require any preference for the noted example. Thus, the disclosure is not to be limited to the examples and designs described herein but is to be accorded the widest scope consistent with the principles and novel features disclosed herein.

1. A method of remote physiological monitoring, comprising:
receiving activity data of a patient from one or more sensors;
receiving physiological data of the patient from one or more sensors;
determining that an at-rest condition is satisfied by at least one of the received activity data and physiological data;
receiving one or more physiological measurements of the patient; and
associating an at-rest indicator with the one or more physiological measurements of the patient based, at least in part, on the determination that an at-rest condition is satisfied.

2. The method of claim 1, wherein receiving activity data comprises:
receiving any one or more of position, velocity, posture, or acceleration data from the one or more sensors.

3. The method of claim 1, wherein receiving physiological data comprises:
receiving any one or more of heart rate, respiration rate, heart rate variability, respiration rate variability, blood pressure, blood oxygen, blood glucose, perspiration, core temperature, electromyography (EMG) data, or electroencephalogram (EEG) data from the one or more sensors.

4. The method of claim 1, further comprising:
receiving environmental data from one or more sensors; and
determining that an at-rest condition is satisfied by any one of the received activity data, physiological data, and environmental data.

5. The method of claim 4, wherein receiving environmental data comprises:
receiving any one or more of temperature, humidity, surface pressure, motion or vibration data from the one or more sensors.

6. The method of claim 1, further comprising:
operating an at-rest timer to measure patient inactivity for a predetermined period of time when the at-rest condition is satisfied; and
associating the received one or more physiological measurements of the patient with an at-rest indicator when the at-rest timer meets or surpasses a predetermined at-rest threshold.

7. The method of claim 1, further comprising:
increasing a frequency of receiving one or more physiological measurements when the patient is determined to be at-rest.

8. The method of claim 1, further comprising:
operating an at-rest timer to measure patient inactivity for a predetermined period of time when the at-rest condition is satisfied; and
operating a glitch timer when, after the at-rest condition is satisfied, at least one of the physiological data and activity data is received and results in a subsequent determination that the at-rest condition is not satisfied.

9. The method of claim 8, further comprising:
continuing operation of the at-rest timer when at least one of the physiological data and activity data has not exceeded a predetermined glitch timer threshold.

10. The method of claim 8, further comprising:
re-initializing the at-rest timer and glitch timer when at least one of the physiological data and activity data has exceeded a predetermined glitch timer threshold.

11. The method of claim 8, further comprising:
setting or adjusting at least one of the at-rest timer duration and glitch timer duration based on patient physiological factors.

12. The method of claim 1, further comprising:
transmitting the received one or more physiological measurements to a monitoring station.

13. The method of claim 1, wherein determining that an at-rest condition is satisfied by at least one of the received activity data and physiological data comprises:
correlating an acceleration signal received with the activity data with a vector magnitude, wherein the vector magnitude is used to calculate a metabolic equivalency (MET).

14. The method of claim 13, further comprising:
determining that the at-rest condition is satisfied when the metabolic equivalency (MET) is 1 based on the activity level of the patient.

15. A remote physiological monitoring device, comprising:
a transceiver configured to receive activity data and physiological data of a patient, and further configured to receive one or more physiological measurements of the patient; and
a processor configured to determine that an at-rest condition is satisfied by at least one of the received activity data and physiological data, and further configured to indicate, based, at least in part, on the determination that an at-rest condition is satisfied, that one or more physiological measurements of the patient were made while the patient was at-rest.

16. The device of claim 15, further comprising:
an at-rest timer configured to measure patient inactivity for a predetermined period of time when the at-rest condition is satisfied.

17. The device of claim 15, further comprising:
a glitch timer configured to operate when, after the processor determines that the at-rest condition is satisfied, at least one of the physiological data and activity data is received and results in a subsequent determination that the at-rest condition is not satisfied.

18. The device of claim 15, further comprising:
a memory configured to record physiological measurements associated with an indicator that the patient is determined to be at-rest.

19. The device of claim 15, wherein the device is portable and is either worn or carried.

20. A non-transitory computer-readable medium storing computer-executable code, the code executable by a processor to:
receive activity data of a patient from one or more sensors;
receive physiological data of the patient from one or more sensors;
determine that an at-rest condition is satisfied by at least one of the received activity data and physiological data;
receive one or more physiological measurements of the patient; and
associate an at-rest indicator with the one or more physiological measurements of the patient based, at least in part, on the determination that an at-rest condition is satisfied.
